(19) United States
(12) Patent Application Publication
(10) Pub. No.: US 2025/0118435 A1
(43) Pub. Date: Apr. 10, 2025

(54) MEDICAL ANALYSIS USING SPATIOTEMPORAL ANALYSIS AND TRANSFORMER-BASED MODELS

(71) Applicant: Emory University, Atlanta, GA (US)

(72) Inventors: Rohan Dhamdhere, Atlanta, GA (US); Sadeer G. Al-Kindi, Houston, TX (US); Gourav Modanwal, Cleveland, OH (US); Anant Madabhushi, Decatur, GA (US)

(21) Appl. No.: 18/901,408

(22) Filed: Sep. 30, 2024

Related U.S. Application Data
(60) Provisional application No. 63/588,471, filed on Oct. 6, 2023.

(51) Int. Cl. G16H 50/20; G06T 7/00; G06V 10/40

(52) U.S. Cl. CPC G16H 50/20; G06T 7/0012; G06V 10/40; G06T 2207/20084

(57) ABSTRACT

The present disclosure relates to a method. The method includes operating a first spatiotemporal model upon a plurality of frames of an anatomic video of a patient to determine a first prediction. The first spatiotemporal model is configured to determine the first prediction using a plurality of hand-crafted features extracted from the plurality of frames. A second spatiotemporal model having one or more deep learning models is operated upon the plurality of frames of the anatomic video to determine a second prediction. A medical prediction is generated based upon a combination of the first prediction and the second prediction.

### REFERENCE TO RELATED APPLICATION

This Application claims the benefit of U.S. Provisional Application No. 63/588,471, filed on Oct. 6, 2023, the contents of which are incorporated by reference in their entirety.

### BACKGROUND

In recent years, there has been significant recent interest in developing computer vision tools for interrogating medical images. Computer vision tools are computer systems that utilize artificial intelligence to analyze images (e.g., medical images). Such systems have the potential to improve health care for patients.

### BRIEF DESCRIPTION OF THE DRAWINGS

The accompanying drawings, which are incorporated in and constitute a part of the specification, illustrate various example operations, apparatus, methods, and other example embodiments of various aspects discussed herein. It will be appreciated that the illustrated element boundaries (e.g., boxes, groups of boxes, or other shapes) in the figures represent one example of the boundaries. One of ordinary skill in the art will appreciate that, in some examples, one element can be designed as multiple elements or that multiple elements can be designed as one element. In some examples, an element shown as an internal component of another element may be implemented as an external component and vice versa. Furthermore, elements may not be drawn to scale.

FIG. 1 illustrates a block diagram of some embodiments of a computer vision assessment system configured to perform a spatiotemporal analysis that combines hand-crafted features and a deep learning model to generate a medical prediction.

FIG. 2 illustrates a block diagram of some additional embodiments of a computer vision assessment system configured to perform a spatiotemporal analysis that combines hand-crafted features and a deep learning model to generate a medical prediction.

FIG. 3 illustrates a block diagram of some additional embodiments of a computer vision assessment system configured to perform a spatiotemporal analysis that combines hand-crafted features and a deep learning model to generate a medical prediction.

FIGS. 4A-4B illustrate graphs showing exemplary hand-crafted features that have prognostic ability to distinguish a medical prediction between different outcomes.

FIG. 5A illustrates exemplary Kaplan-Meier graphs showing a predicted overall survival for different biomarkers.

FIG. 5B illustrates a table showing exemplary performance parameters for different biomarkers.

FIG. 6 illustrates some embodiments of a flow diagram showing a method for performing a spatiotemporal analysis that combines hand-crafted features and deep learning models to generate a medical prediction.

FIG. 7 illustrates a flow diagram corresponding to a method of making a medical prediction for a major adverse cardiovascular event (MACE) using spatiotemporal analysis that combines radiomic features and a deep learning model.

FIG. 8 illustrates a block diagram of some embodiments of a medical analysis system comprising a computer vision assessment system configured to perform a spatiotemporal analysis that combines hand-crafted features and a deep learning model to generate a medical prediction.

### DETAILED DESCRIPTION

The description herein is made with reference to the drawings, wherein like reference numerals are generally utilized to refer to like elements throughout, and wherein the various structures are not necessarily drawn to scale. In the following description, for purposes of explanation, numerous specific details are set forth in order to facilitate understanding. It may be evident, however, to one of ordinary skill in the art, that one or more aspects described herein may be practiced with a lesser degree of these specific details. In other instances, known structures and devices are shown in block diagram form to facilitate understanding.

Cardiovascular diseases are diseases that affect a person's heart and/or blood vessels. They are the leading cause of death both worldwide and in the United States. Cardiovascular diseases are especially dangerous for patients with chronic kidney disease (CKD). For example, cardiovascular disease accounts for between approximately 40% and 50% of deaths in patients with acute CKD. Because CKD patients have a high likelihood of cardiovascular disease events, CKD patient management often includes regular prognostic analysis to try to determine if a patient is likely to experience a major adverse cardiovascular event (MACE).

Echocardiography is a non-invasive imaging modality that provides a quick assessment of cardiac structure and function for cardiovascular disease diagnosis. Despite its usefulness, echocardiography data is often noisy and has a relatively poor resolution. The noise and poor resolution cause challenges in effectively interpreting and analyzing echocardiography data. Measurements like left ventricle (LV) volume captured by ejection fraction (EF), LV mass, and LV geometry have been standard biomarkers for diagnosing and predicting a severity of cardiovascular disease. However, standard echocardiography measurements based on static LV volume and morphology (e.g., such as EF) may have limited prognostic value beyond baseline clinical characteristics as adverse outcomes are a common occurrence in spite of a preserved EF.

It has been appreciated that non-static anatomic measurements from medical images may provide for non-traditional biomarkers that can improve disease forecasting. For example, left ventricle wall (LVW) alterations may be a non-traditional biomarker of cardiovascular disease in CKD patients due to pathophysiological changes in the LVW. Abnormalities of the LVW movement may also be a prognostic biomarker for MACE prediction in cardiovascular disease patients. For example, longitudinal dysfunction, which is common in CKD patients, is reflected in LVW with change in morphology. This dysfunction has been associated with CKD progression and abnormalities are evident even in early stages of CKD.

The present disclosure relates to a computer vision assessment system that is configured to perform a spatiotemporal analysis of an anatomic video to generate a biomarker that can be used to generate a medical prediction. The computer vision assessment system may perform the spatiotemporal analysis using a combination of hand-crafted features (e.g., radiomic features aggregated from differential spatial regions over time) and one or more deep learning models. Hand-crafted features are able to model cardiac morphology for predicting disease outcomes, while deep learning models may be used for modelling of spatiotemporal changes in anatomy (e.g., a left ventricle). Thus, a combination of radiomic and deep learning models could associate anatomic changes over time with disease progression and thereby potentially provide some insight into the factors implicated for disease outcomes.

In some embodiments, the disclosed computer vision assessment system may be configured to perform a spatiotemporal analysis by accessing an echocardiography video of a patient. The echocardiography video has a plurality of frames spanning a time. The echocardiography video is operated upon by a first spatiotemporal model that is configured to determine a first prediction from a first set of hand-crafted features extracted over the plurality of frames. The echocardiography video is further operated upon by a second spatiotemporal model, which comprises one or more deep learning models, to determine a second prediction. The first prediction and the second prediction are both used to generate a medical prediction. By combining the predictions of the first and second spatiotemporal models, the method is able to analyze a heart's morphology (e.g., a morphology of a left ventricle wall) over time, thereby improving prediction of major adverse cardiovascular event (MACE) risk in a patient (e.g., in a CKD patient).

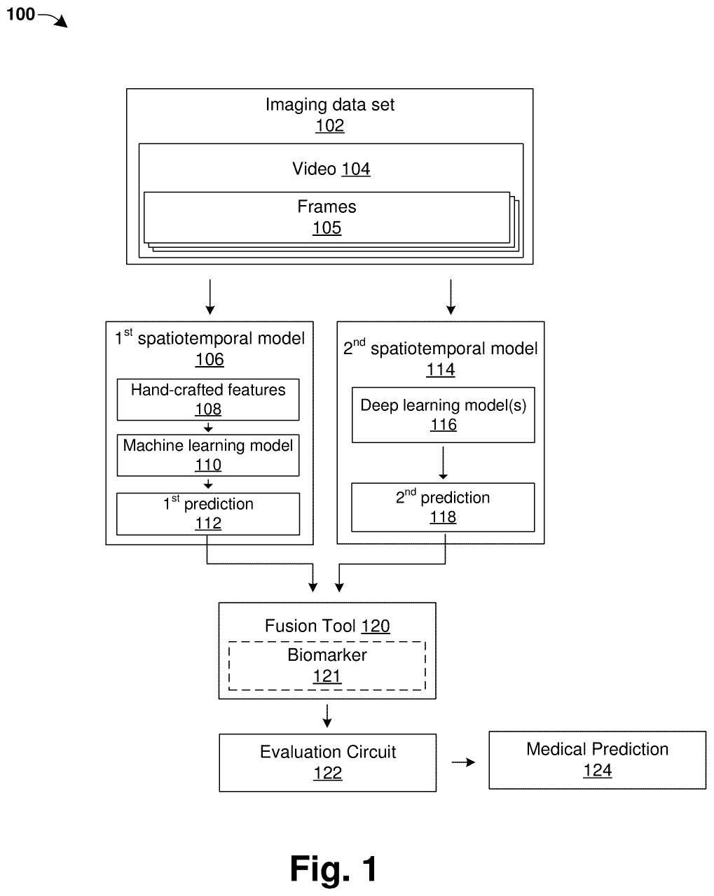

FIG. 1 illustrates a block diagram of some embodiments of a computer vision assessment system 100 configured to perform a spatiotemporal analysis that combines hand-crafted features and a deep learning model to generate a medical prediction.

The computer vision assessment system 100 comprises an imaging data set 102 including an anatomic video 104 of a patient. The anatomic video 104 comprises a plurality of frames 105 that span a time period. In some embodiments, the anatomic video 104 may comprise an echocardiography video of a patient's heart. In such embodiments, the echocardiography video may comprise a plurality of frames 105 that span a time period that is equal to a heartbeat cycle of the patient. In other embodiments, the anatomic video 104 may comprise a video of the patient's brain, one or more lungs, etc.

In some embodiments, the plurality of frames 105 may comprise frames that have been segmented to identify a specific region of interest (ROI) within an anatomy. For example, in some embodiments the plurality of frames 105 may comprise frames that have been segmented to identify an ROI that includes a left ventricle wall (LVW) of a heart of the patient. In other embodiments, the plurality of frames 105 may comprise frames that have been segmented to identify an ROI including other regions of a heart (e.g., a right ventricle wall), regions of a brain, regions of a lung, etc.

The plurality of frames 105 are provided to a first spatiotemporal model 106. The first spatiotemporal model 106 is configured to extract a plurality of hand-crafted features 108 from each of the plurality of frames 105 of the anatomic video 104. The plurality of hand-crafted features 108 describe both spatial relationships within the plurality of frames 105 and temporal relationships between the plurality of frames 105. For example, the plurality of hand-crafted features 108 may include radiomic features that describe spatiotemporal changes in the anatomy of the patient. The plurality of hand-crafted features 108 are operated upon by a machine learning model 110 to generate a first prediction 112 for the patient.

The plurality of frames 105 are further provided to a second spatiotemporal model 114. The second spatiotemporal model 114 comprises one or more deep learning models 116 that are configured to perform both spatial and temporal encoding on the plurality of frames 105 to generate a second prediction 118 for the patient. The first prediction 112 and the second prediction 118 may correspond to a same issue (e.g., a risk of a MACE), but may have different parameters and/or values.

In some embodiments, a fusion tool 120 is configured to combine the first prediction 112 and the second prediction 118 to generate a biomarker 121. The biomarker 121 may be provided to an evaluation tool 122, which is configured to generate a medical prediction 124 for the patient. In other embodiments, the fusion tool 120 and the evaluation tool 122 may comprise a single tool (e.g., a single machine learning model configured receive the first prediction 112 and the second prediction 118 as inputs and to generate the medical prediction 124). By using both the first prediction 112 and the second prediction 118 to generate the medical prediction 124, the computer vision assessment system 100 is able to accurately analyze a ROI over time, thereby improving an accuracy of the medical prediction 124.

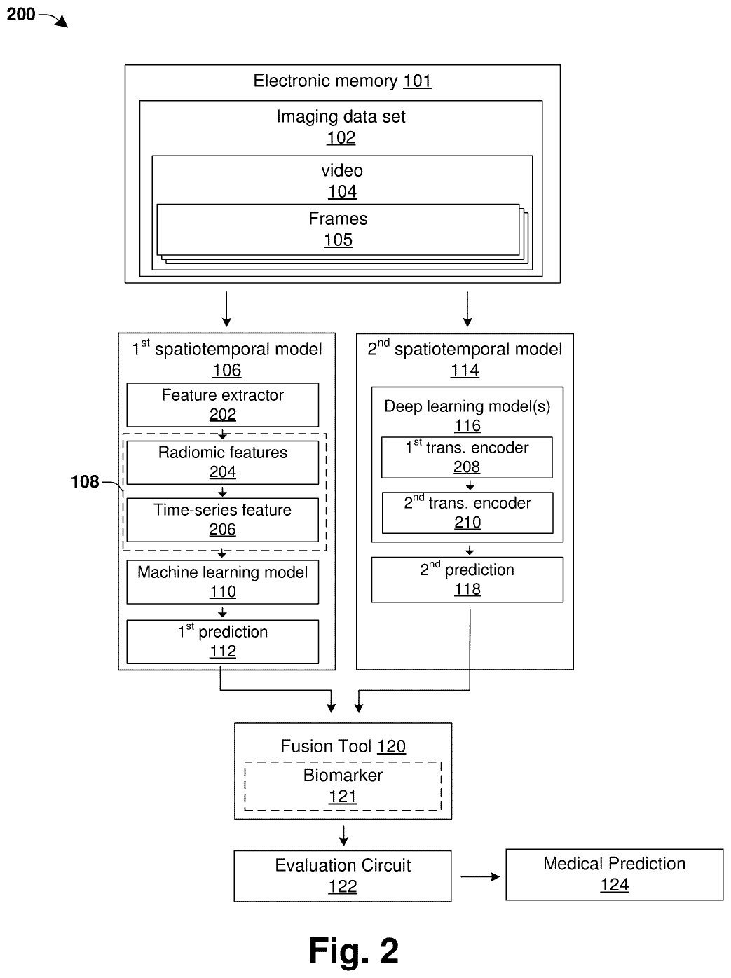

FIG. 2 illustrates a block diagram of some additional embodiments of a computer vision assessment system 200 configured to perform a spatiotemporal analysis that combines hand-crafted features and a deep learning model to generate a medical prediction.

The computer vision assessment system 200 comprises an imaging data set 102 including an anatomic video 104 of a patient. The anatomic video 104 comprises a plurality of frames 105 that span a time period (e.g., a heartbeat cycle of the patient). In some embodiments, the plurality of frames 105 may comprise two-dimensional frames respectively taken at a given time (e.g., a first frame taken at a first time, a second frame taken at a second time, etc.). In some embodiments, the plurality of frames 105 may comprise frames that have been segmented to identify a region of interest (ROI). In some embodiments, the imaging data set 102 and the anatomic video 104 may be stored in electronic memory 101. In various embodiments, the electronic memory 101 may comprise read-only memory (ROM), random-access memory (RAM), erasable programmable read-only memory (EPROM), electrically erasable programmable read-only memory (EEPROM), flash memory, dynamic random-access memory (DRAM), static random-access memory (SRAM), and/or the like.

The plurality of frames 105 are provided to a first spatiotemporal model 106. The first spatiotemporal model 106 comprises a feature extractor 202 configured to extract a plurality of radiomic features 204 from each of the plurality of frames 105. The plurality of radiomic features 204 may comprise shape features, texture features (e.g., gray-level features), and first order statistics of the ROI in each of the plurality of frames 105. A time-series feature 206 is generated from radiomic features extracted from multiple ones of the plurality of frames 105. The time-series feature 206 (e.g., which also includes information of the plurality of radiomic features) is provided to a machine learning model 110 (e.g., a random forest classifier, a regression model, etc.), which is configured to operate upon the time-series feature 206 to determine a first prediction 112 for the patient. In some embodiments, the first prediction 112 may comprise a first numeric risk score corresponding to an issue (e.g., a MACE event).

In some embodiments, the feature extractor 202 may comprise a pyradiomics python package implemented as computer code run by a processing unit (e.g., a central processing unit including one or more transistor devices configured to operate computer code to achieve a result, a microcontroller, or the like). In some embodiments, the time-series feature 206 may be extracted from the plurality of radiomic features 204 for the plurality of frames 105 using a TSFresh python library implemented as computer code run by a processing unit. In some embodiments, the machine learning model 110 may comprise code run by a processing unit.

The plurality of frames 105 are further provided to a second spatiotemporal model 114 configured to determine a second prediction 118 for the patient using one or more deep learning models 116. In some embodiments, the second prediction 118 may comprise a second numeric risk score corresponding to an issue (e.g., a MACE event). In some embodiments, the second spatiotemporal model 114 may comprise a transformer model having one or more transformer encoders 208-210 (e.g., the second spatiotemporal model 114 may comprise a video vision transformer model having a factorized encoder). In such embodiments, the plurality of frames 105 may be embedded into a plurality of tokens. The one or more transformer encoders 208-210 are configured to determine a spatial relationship between tokens from a same frame of the plurality of frames 105 and to determine one or more temporal relationships between the plurality of frames 105. For example, in some embodiments the second spatiotemporal model 114 comprises a first transformer encoder 208 and a second transformer encoder 210 arranged in series downstream of the first transformer encoder 208. The first transformer encoder 208 is configured to determine a spatial relationship between tokens from a same frame of the plurality of frames 105. The second transformer encoder 210 is configured to determine one or more temporal relationships between tokens from different ones of the plurality of frames 105.

A fusion tool 120 is configured to combine the first prediction 112 and the second prediction 118 to generate a biomarker 121. In some embodiments, the fusion tool 120 may combine the first prediction 112 and the second prediction 118 using linear discriminant analysis (LDA) based fusion. The biomarker 121 may be provided to an evaluation tool 122, which is configured to generate a medical prediction 124 (e.g., a numeric value indicating a prediction of a MACE) for the patient. In some embodiments, the fusion tool 120 and the evaluation tool 122 may comprise a single tool (e.g., a single machine learning model configured receive the first prediction 112 and the second prediction 118 as inputs and to generate the medical prediction 124).

In some embodiments, the medical prediction 124 may comprise a numeric value indicating a probability of an occurrence of a cardiac event (e.g., a major adverse cardiovascular event (MACE) such as chronic heart failure, a myocardial infraction, a stroke, and/or the like.). In some embodiments, the patient may be a cardiovascular disease patient (e.g., a patient having or suspected of having cardiovascular disease) that also has chronic kidney disease (CKD). Since patients with CKD have a higher risk of MACE, the computer vision assessment system 200 may have a good applicability to patients with CKD since such patients are more susceptible to MACE. In other embodiments, the medical prediction 124 may comprise a numeric value corresponding to a brain disease (e.g., an aneurysm, inflammation, neurodegenerative diseases such as Alzheimer's and dementia, Parkinson's diseases, etc.), a pulmonary disease (e.g., lung cancer, pneumonia, etc.), allograft rejection (e.g., heart transplant rejection), and/or a similar disease outcome.

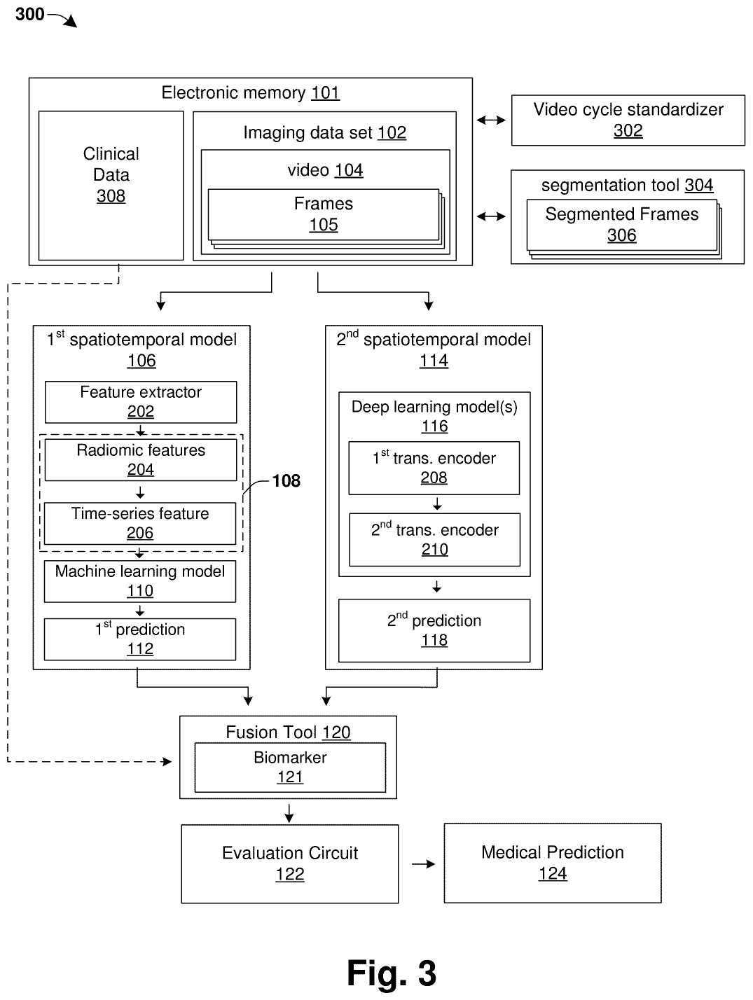

FIG. 3 illustrates a block diagram of some additional embodiments of a computer vision assessment system 300 configured to perform a spatiotemporal analysis that combines hand-crafted features and a deep learning model to generate a medical prediction.

The computer vision assessment system 300 comprises an imaging data set 102 stored in electronic memory 101. The imaging data set 102 includes an anatomic video 104 of a patient. In some embodiments, the anatomic video 104 may comprise an echocardiography video including a plurality of frames 105 that span a time period (e.g., a heartbeat cycle of the patient). For example, a video V may comprise N frames (e.g., V=[I₁, I₂, . . . , I_{N}]). In some embodiments, the plurality of frames 105 may comprise two-dimensional frames respectively taken at a given time and including a left ventricle wall (LVW) of a heart of the patient.

In some embodiments, a video cycle standardizer 302 may be configured to adjust a number of frames within the time period of the anatomic video 104, so that different anatomic videos from different patients have a standard number of frames (e.g., 30 frames). For example, it has been appreciated that heart-beat cycle frame lengths may vary between individual patients. Therefore, to achieve a standard frame length (e.g., of 30 frames) the video cycle standardizer 302 may be configured to drop random frames from an echocardiography video based on normal distribution from videos with extra frames (e.g., with a number of frames larger than the standard frame length), while in videos with fewer frames (e.g., with a number of frames smaller than the standard frame length) the video cycle standardizer 302 may be configured to generate interpolated frames between randomly selected adjacent frames. In some embodiments, the video cycle standardizer 302 may determine a heart-beat cycle using consecutive end-diastolic phases identified by a CNN+LSTM (convolutional neural network (CNN) with a long short-term memory (LSTM) layer) based multi-beat echo phase detection model.

In some embodiments, a segmentation tool 304 is configured to segment the plurality of frames 105 of the anatomic video 104 to identify a region of interest (ROI) within each of the plurality of frames 105. For example, the segmentation tool 304 may segment a plurality of frames within an echocardiography video to identify a left ventricle wall (LVW) of a heart within each of the plurality of frames. In some embodiments, the segmentation tool 304 is configured to mask the plurality of frames 105 to form a plurality of segmented frames 306 (e.g., a 30-frame sequence of masked LVW image frames). In some embodiments, the plurality of segmented frames 306 may be provided back to the electronic memory 101 and saved as the plurality of frames 105. In other embodiments (not shown), the plurality of segmented frames 306 may be provided downstream (e.g., to first and second spatiotemporal models). In some embodiments, the segmentation tool 304 may be further configured to crop (e.g., center-crop) each frame and resize each cropped frame. In some embodiments, the segmentation tool 304 may comprise a convolutional neural network (e.g., a U-NET, nnU-Net, or the like).

The plurality of frames 105 are provided to a first spatiotemporal model 106. The first spatiotemporal model 106 comprises a feature extractor 202 configured to extract a plurality of hand-crafted features 108 from the plurality of frames 105. In some embodiments, the plurality of hand-crafted features 108 may describe spatiotemporal changes between the plurality of frames 105. For example, the plurality of hand-crafted features 108 may describe how extracted features change within a space of frames over time.

In some embodiments, the plurality of hand-crafted features 108 may comprise a plurality of radiomic features 204 comprising shape features, texture features (e.g., gray-level features), and first order statistics of the ROI in each of the N frames (e.g., R(I_{N})=f(I_{N}), where f is a function of feature extraction and N is a frame number). In some embodiments, the radiomic features 204 may be related to a LVW. For example, in some embodiments the plurality of radiomic features 204 may comprise a sphericity of a LVW, a shape based perimeter of a LVW, a texture based feature of a LVW, and/or the like. The plurality of hand-crafted features 108 may further comprise a time-series feature 206 generated from the plurality of radiomic features 204 of each of the plurality of frames 105 (e.g., the time-series feature 206 of a video includes radiomic features extracted from each frame of the video, t_{R}(V)−[R(I₁), R(I₂), . . . R(I_{N})]). The time-series feature 206 comprises information relating to the spatiotemporal evolution of the plurality of radiomic features 204 (e.g., how the sphericity, perimeter, and/or texture features change over space and time).

The time-series feature 206 is provided to a machine learning model 110 (e.g., a random forest classifier, a regression model, etc.). The machine learning model 110 is configured to operate upon the time-series feature 206 to determine a first prediction 112 for the patient. In some embodiments, the first spatiotemporal model 106 may be configured to perform feature selection to identify features having a high prognostic value. For example, in some embodiments the machine learning model 110 may utilize a minimum redundancy maximum relevant (mRMR) method, a Lasso (least absolute shrinkage and selection operator) method, a Boruta algorithm, and/or the like, to select prognostic features.

The plurality of frames 105 are further provided to a second spatiotemporal model 114 configured to determine a second prediction 118 for the patient using one or more deep learning models 116. In some embodiments, the second spatiotemporal model 114 comprises a first transformer encoder 208 and a second transformer encoder 210 arranged in series downstream of the first transformer encoder 208. The first transformer encoder 208 is configured to determine a spatial relationship between tokens from a same frame of the plurality of frames 105. The second transformer encoder 210 is configured to determine one or more temporal relationships between the plurality of frames 105.

A fusion tool 120 is configured to combine the first prediction 112 and the second prediction 118 to generate a biomarker 121 corresponding to the patient. In some embodiments, the fusion tool 120 may combine the first prediction 112 and the second prediction 118 to form the biomarker 121 using linear discriminant analysis (LDA) based fusion. For example, the fusion tool 120 may add a first prediction (e.g., M_{R}(T(R(I_{N})ᵢ) and a second prediction (e.g., M_{T}(Vᵢ)) to generate the biomarker 121 (e.g., Xᵢ=M_{R}(T(R(I_{N})ᵢ+M_{T}(Vᵢ)). In some embodiments, the biomarker 121 may characterize a heart's morphology (e.g., a morphology of a left ventricle wall) over time, so as to provide for information about changes in structure and/or features over time.

In some embodiments, the fusion tool 120 may also be configured to include clinical data 308 of the patient into the biomarker 121. For example, clinical data 308 from the patient may be stored in the electronic memory 101. The fusion tool 120 may be configured to receive the clinical data 308 from the electronic memory 101 and to combine the clinical data 308 with the first prediction 112 and the second prediction 118 to generate the biomarker 121. In various embodiments, the clinical data 308 may include demographic information (e.g., age, race, sex, etc.), height, weight, blood pressure, cholesterol, and/or the like, of the patient.

The biomarker 121 is provided to an evaluation tool 122. The evaluation tool 122 is configured to utilize the biomarker 121 to generate a medical prediction 124. In some embodiments, the fusion tool 120 and the evaluation tool 122 may comprise a single machine learning model configured receive the first prediction 112, the second prediction 118, and the clinical data 308 as inputs. In some embodiments, the evaluation tool 122 may generate the medical prediction 124 to be a probability of a MACE. Because the biomarker 121 is able to characterize a heart's morphology over time, it enables an improved prediction of MACE risk in a patient (e.g., in a CKD patient).

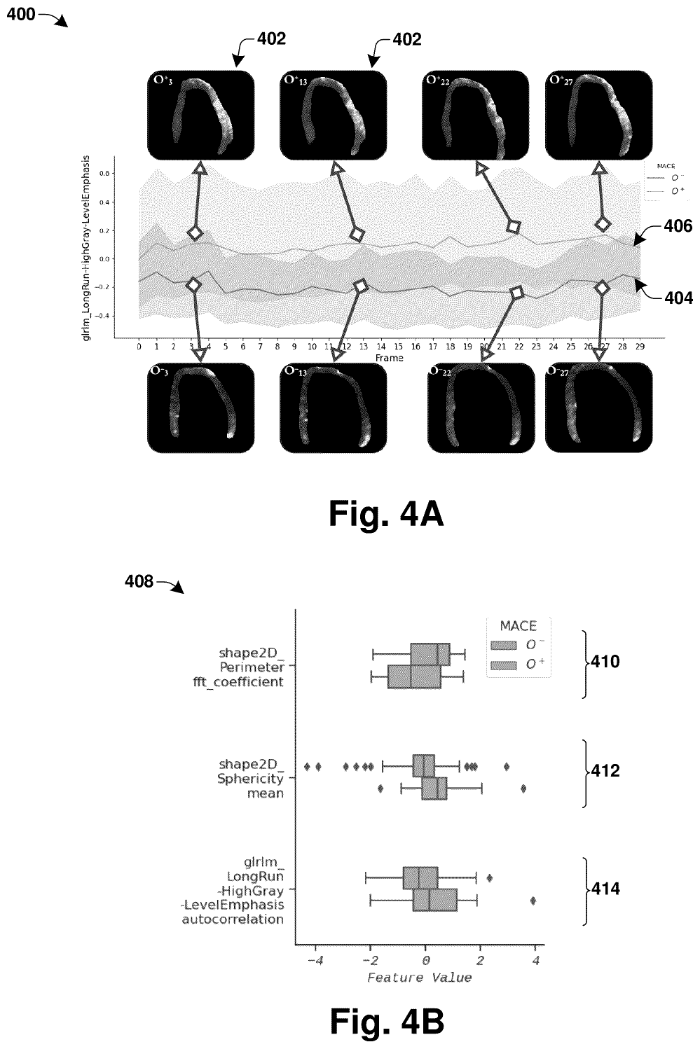

FIG. 4A illustrates a graph 400 illustrating exemplary radiomic feature values extracted from anatomic videos.

As shown in graph 400, anatomic videos respectively comprise a plurality of frames (shown along x-axis). In some embodiments, the anatomic videos may comprise 30 frames (e.g., frames 0-29 are shown along x-axis), while in other embodiments (not shown) the anatomic videos may comprise more or less than 30 frames. Within each of the plurality of frames, a region of interest (ROI) 402 (e.g., a left ventricle wall (LVW)) is identified and then values of one or more radiomic features are extracted from the ROI 402 (shown along y-axis). For example, for each frame a gray-level radiomic feature may be determined from statistical analysis that quantifies the spatial relationships of pixel intensities within a LVW.

As can be seen in graph 400, the values of the shape and texture radiomic features within the ROI may vary from frame to frame, thereby showing how the LVW changes over time. Furthermore, the values of the radiomic feature (e.g., texture features illustrated by changing colors corresponding to pixel intensity and shape features) may be different between patients 404 that are predicted to experience a MACE and patients 406 that are predicted to not experience a MACE.

FIG. 4B illustrates a graph 408 showing exemplary box plots associated with radiomic features that show a high prognostic ability to determine a MACE.

As shown in FIG. 4B, a first pair of box plots 410 are shown for a radiomic feature corresponding to longitudinal changes in a perimeter of a LVW shape, a second pair of box plots 412 are shown for a radiomic feature corresponding to changes in sphericity of a LVW shape, and a third pair of box plots 414 are shown for a radiomic feature corresponding to a gray-level texture feature of a LVW shape. For each of the radiomic features, the box plots show differences in feature values for patients that are predicted to experience a MACE and patients that are predicted to not experience a MACE, thereby showing that the radiomic features are prognostic of a MACE.

It will be appreciated that the use of hand-crafted features (e.g., radiomic features) within the disclosed computer vision assessment system provides for interpretability with regard to the underlying physical circumstances. For example, changes in the LVW shape can indicate left ventricular hypertrophy (LVH), a common complication of CKD linked to a higher risk of cardiovascular events. LVH can stem from various factors that modify the ventricle's structure and geometry. Texture changes in LV may also reflect alterations in collagen content or fibrosis which increase the risk of adverse events associated with cardiac remodeling. The interpretability of the hand-crafted features may allow for other prognostic hand-crafted features to be manually identified (e.g., if a feature is found to be prognostic, similar features can be explored). Furthermore, the interpretability of the hand-crafted features may also provide medical professionals with an underlying physical basis for a medical prediction that can improve confidence in the medical prediction.

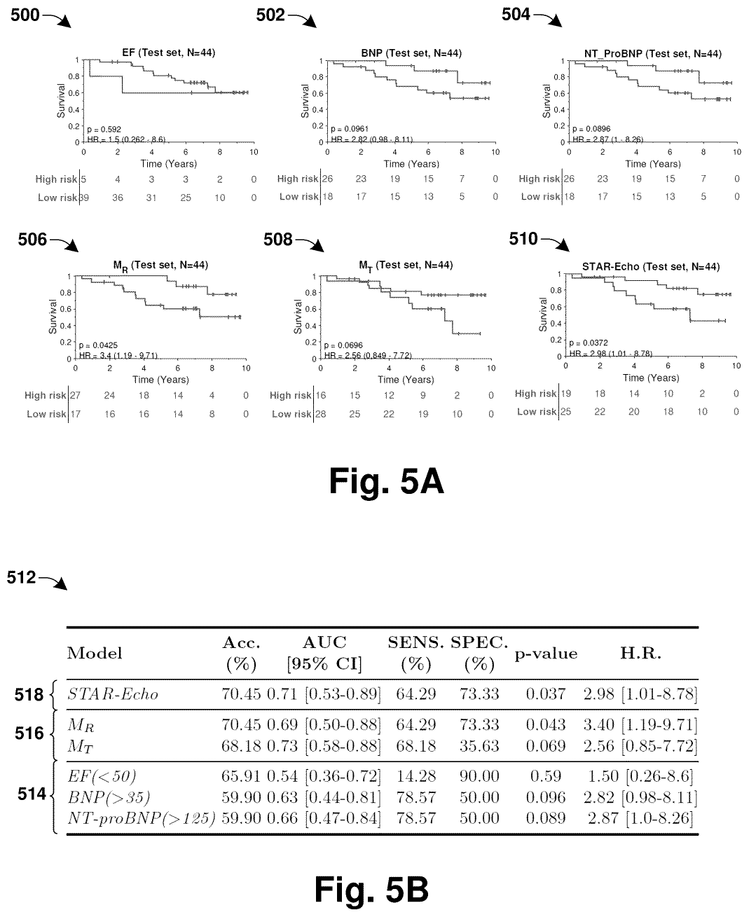

It has also been appreciated that the use of both the hand-crafted features and the one or more deep learning models to generate the biomarker provides the disclosed computer vision assessment system with an improved performance over other biomarkers (e.g., including the use of one of either hand-crafted features or deep learning models). For example, FIGS. 5A-5B illustrate a plurality of different metrics that characterize a performance of the disclosed computer vision assessment system in comparison to other systems.

FIG. 5A illustrates exemplary Kaplan-Meier graphs 500-510 showing a predicted overall survival for different biomarkers.

As shown in FIG. 5A, Kaplan-Meier graphs 500-510 show Kaplan-Meier curves corresponding to an overall survival predicted using different biomarkers. The first graph 500 corresponds to a computer vision assessment system that generates a medical prediction by operating upon a biomarker comprising an ejection factor (EF). The second graph 502 corresponds to a computer vision assessment system that generates a medical prediction by operating upon a biomarker comprising a B-type natriuretic peptide (BNP). The third graph 504 corresponds to a computer vision assessment system that generates a medical prediction by operating upon a biomarker comprising an N-terminal pro-B-type natriuretic peptide (NT-proBNP). The fourth graph 506 corresponds to a computer vision assessment system that generates a medical prediction by operating upon a biomarker comprising radiomic features extracted from an anatomic video. The fifth graph 508 corresponds to a computer vision assessment system that generates a medical prediction by operating a deep learning transformer model on an anatomic video. The sixth graph 510 corresponds to a disclosed computer vision assessment system.

For each of the Kaplan-Meier graphs 500-510, the x-axis represents a time in months and y-axis represents the estimated survival probability. The Kaplan-Meier graphs 500-510 respectively have a high-risk stratification group (red line) and a low-risk stratification group (blue line). In some embodiments, stratification between the high-risk stratification group and the low-risk stratification group may be based on a median of risk scores generated during training of an associated system.

As can be seen in the KM graphs 500-510, the sixth graph 510 (associated with the disclosed computer vision assessment system) achieves a good stratification between the high-risk stratification group and the low-risk stratification group. Furthermore, the sixth graph is able to achieve both a high hazard ratio (e.g., HR=2.98, with a range of between 1.01 and 8.78) and a low p-value (e.g., 0.0372), thereby indicating that the disclosed computer vision assessment system has a good prognostic ability.

FIG. 5B illustrates a table 512 showing exemplary performance parameters for different biomarkers.

Table 512 shows an accuracy, an area under an ROC curve (AUC), a sensitivity, a specificity, a p-value, and a hazard ratio for computer vision systems using clinical based biomarkers 514, computer vision systems using machine learning biomarkers 516, and a disclosed computer vision systems using a biomarker 518 generated from a combination of predictions determined from hand-crafted features and one or more deep learning models. As shown in table 512, the disclosed computer vision assessment system is able to achieve a relatively high AUC (e.g., 0.71) and a high accuracy (e.g., 70.45%) with a small p-value (e.g., 0.037). The combination of high AUC and small p-value indicates that the disclosed computer vision assessment system is able to achieve improved performance over other conventional systems.

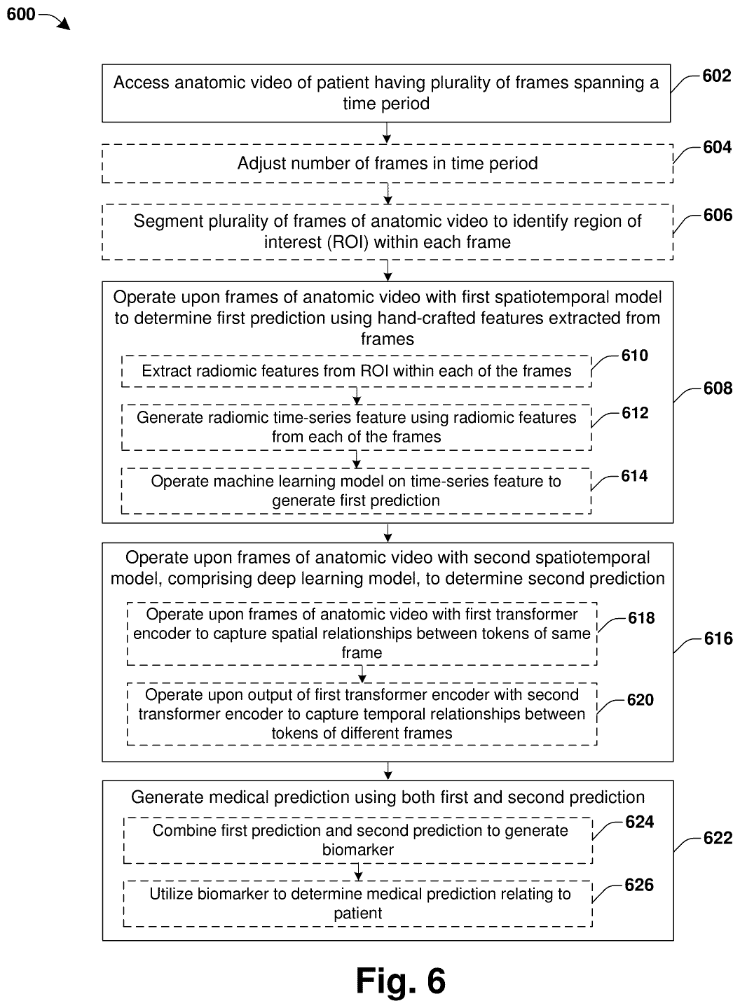

FIG. 6 illustrates some embodiments of a flow diagram showing a method 600 for combining spatiotemporal radiomics and transformer-based models to generate a medical prediction relating to MACE.

While the disclosed method 600 is illustrated and described herein as a series of acts or events, it will be appreciated that the illustrated ordering of such acts or events are not to be interpreted in a limiting sense. For example, some acts may occur in different orders and/or concurrently with other acts or events apart from those illustrated and/or described herein. In addition, not all illustrated acts may be required to implement one or more aspects or embodiments of the description herein. Further, one or more of the acts depicted herein may be carried out in one or more separate acts and/or phases.

At act 602, an anatomic video of a patient is accessed. The anatomic video comprises a plurality of frames spanning a time period. In some embodiments, the anatomic video comprises an echocardiography video having a plurality of frames spanning a time period equal to a heart-beat cycle. In some embodiments, the patient may be a cardiovascular disease patient having chronic kidney disease.

At act 604, a number of frames in the time period (e.g., the heart-beat cycle) may be adjusted (e.g., increased or decreased).

At act 606, the plurality of frames of the anatomic video may be segmented to form identify a region of interest (ROI) within each frame. In some embodiments, the ROI may comprise a left ventricle wall (LVW).

At act 608, the plurality of frames of the anatomic video are operated upon by a first spatiotemporal model configured to determine a first prediction from a plurality of hand-crafted features extracted from the plurality of frames. In some embodiments, the first spatiotemporal model is configured to determine the first prediction according to acts 610-614.

At act 610, a plurality of radiomic features are extracted from a ROI within each of the plurality of frames.

At act 612, a time-series feature is generated using the plurality of radiomic features extracted from each of the plurality of frames. The time-series feature includes information from the plurality of radiomic features over the plurality of frames.

At act 614, a machine learning model is operated upon the time-series feature to generate the first prediction.

At act 616, the plurality of frames of the anatomic video are operated upon with a second spatiotemporal model, comprising one or more deep learning models (e.g., transformer models), which are configured to determine a second prediction. In some embodiments, the second spatiotemporal model is configured to determine the second prediction according to acts 618-620.

At act 618, a first transformer encoder is operated upon the plurality of frames of the anatomic video to capture spatial relationships between tokens of a same frame.

At act 620, a second transformer encoder is operated upon outputs of the first transformer encoder to capture one or more temporal relationships between tokens of different frames.

At act 622, a medical prediction is generated using both the first prediction and the second prediction. In some embodiments, the medical prediction may be generated according to acts 624-626.

At act 624, the first prediction and the second prediction are combined to generate a biomarker.

At act 626, the biomarker is utilized to determine a medical prediction relating to the patient.

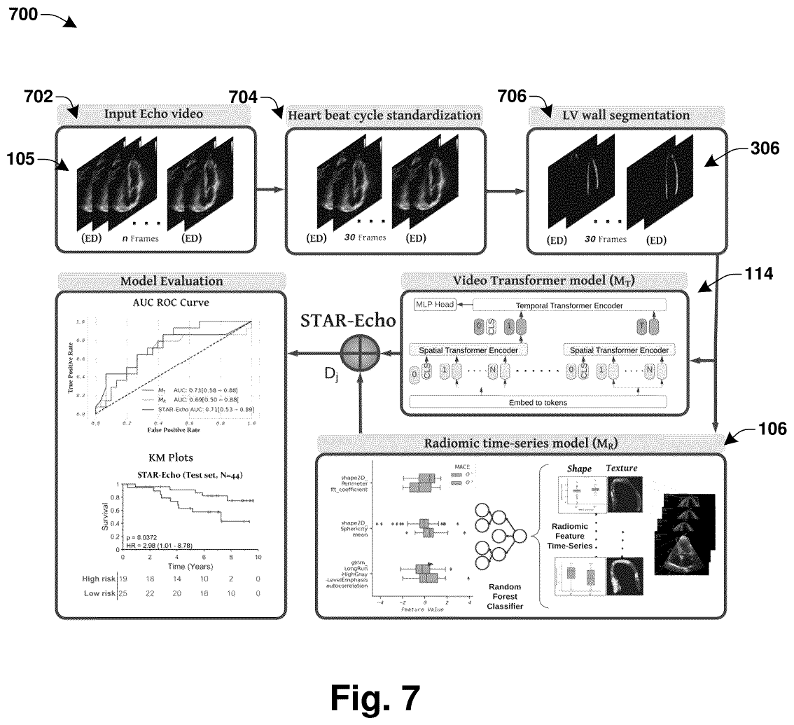

FIG. 7 illustrates a flow diagram 700 corresponding to a method of making a medical prediction for a major adverse cardiovascular event (MACE) using spatiotemporal analysis that combines radiomic features and a deep learning model.

As shown in flow diagram 700, an echocardiography video 702 is accessed. The echocardiography video 702 may have a plurality of frames 105 (e.g., the echocardiography video 702 may have n frames, wherein n equals 30, 35, 40, or other similar values). In some embodiments, the echocardiography video 702 may be accessed from a data storage device, including a hard disk drive, a solid state device, a tape drive, over a local area network, over the internet, and/or the like.

A heart-beat cycle standardization procedure 704 may be performed to standardize a number of frames within the echocardiography video 702. Because heart-beat cycle frame lengths may vary with individual patients, the heart-beat cycle standardization procedure 704 may achieve a standard frame length (e.g., of 30 frames) for echocardiography videos. In some embodiments, the heart-beat cycle standardization procedure 704 may achieve a standard frame length by randomly dropping frames based on normal distribution in echocardiography videos with extra frames, and using video-frame interpolation model to generate frames between randomly selected adjacent frames while in echocardiography videos with fewer frames. In some embodiments, the heart-beat cycle standardization procedure 704 may determine a heart-beat cycle using a consecutive end-diastolic phases identified by a CNN+LSTM (convolutional neural network (CNN) with a long short-term memory (LSTM) layer) based multi-beat echo phase detection model.

A segmentation procedure 706 is configured to perform a segmentation process (e.g., a weakly supervised segmentation approach) to generate a plurality of segmented frames 306 that identify a left ventricle wall (LVW) for frames of the echocardiography video 702. The segmentation procedure 706 may be done by a trained an nnUNet-based U-Net model. In some embodiments, the segmentation procedure 706 may mask the Echocardiography video with a mask to provide a 30-frame sequence of masked LVW image frames. Additionally, each image frame may be center-cropped and resized to equal k×k dimensions (e.g., so that the video has dimensions of 30×k×k).

The plurality of segmented frames 306 may be input to a first spatiotemporal model 106 (M_{R}) and a second spatiotemporal model 114 (M_{T}). In some embodiments, the first spatiotemporal model 106 may comprise a radiomic time-series model. In some embodiments, the second spatiotemporal model 114 may comprise a video transformer model with a factorized encoder. For example, the second spatiotemporal model 114 may comprise a video vision transformer model having a factorized encoder and operating with hyperparameters. In some embodiments, the hyperparameters may including a batch size of 2, 50 epochs, a learning rate of 3× e³, an input size of 256×256 units (e.g., pixels), a patch size of 8×8 units, 9 layers, 7 heads, and a dropout % of 20%.

The first spatiotemporal model 106 is configured to employ a two-stage feature extraction process to extract a plurality of hand-crafted features from the plurality of segmented frames 306. In a first stage, radiomic features are extracted from the LVW for each frame of the plurality of segmented frames 306. In some embodiments, a total of 97 radiomic features are extracted per frame of the echocardiography video. In a second stage, to model the temporal LVW motion, a sequence of radiomic features from each phase of one heartbeat cycle is provided as a time-series feature (e.g., a progression of radiomic feature values corresponding to radiomic features within the different segmented frames). A time-series feature is extracted for each radiomic feature time-series using the TSFresh python library.

The time-series feature is provided to a regression model (e.g., a Cox regression model), which is configured to generate a first prediction. In some embodiments, a feature selection operation may be performed to select a plurality of the features that are most prognostic features (e.g., a group of the 3 most prognostic features may be selected from the 97 radiomic feature extracted per frame). In some embodiments, the feature selection operation may utilize a minimum redundancy maximum relevant (mRMR) method, a Lasso (least absolute shrinkage and selection operator) method, a Boruta algorithm model (e.g., a random forest classifier), and/or the like.

The plurality of segmented frames 306 are also provided to the second spatiotemporal model 114. In some embodiments, an architecture of the second spatiotemporal model 114 may comprise a plurality of transformer encoders. In some such embodiments, the plurality of segmented frames 306 and/or fragments of the plurality of segmented frames 306 may be provided to a first transformer encoder as one or more tokens (e.g., embedded vectors corresponding to different parts of an ROI). In some such embodiments, an embedding layer may be configured to project pixel values within the different ROI onto the one or more tokens.

In some embodiments, an architecture of the second spatiotemporal model 114 may have two transformer encoders connected in series. The first encoder captures the spatial relationships between tokens from the same frame and generates a hidden representation for each frame. The second encoder captures one or more temporal relationships between frames, resulting in a late fusion of temporal and spatial information.

The first prediction and the second prediction are combined using linear discriminant analysis (LDA) based fusion and then used to generate a medical prediction (e.g., a probability) using a machine learning model having a plurality of learned parameters.

In some embodiments, the radiomic time-series model M_{R} may be trained on time-series features T_{R}(V) obtained from videos (e.g., masked with LVW) of a plurality of patients and the second spatiotemporal model 114 may be trained (e.g., to tune hyperparameters) using the videos (e.g., masked with LVW) of the plurality of patients to generate the medical prediction.

It will be appreciated that the disclosed methods and/or block diagrams may be implemented as computer-executable instructions, in some embodiments. Thus, in one example, a computer-readable storage device (e.g., a non-transitory computer-readable medium) may store computer executable instructions that if executed by a machine (e.g., computer, processor) cause the machine to perform the disclosed methods and/or block diagrams. While executable instructions associated with the disclosed methods and/or block diagrams are described as being stored on a computer-readable storage device, it is to be appreciated that executable instructions associated with other example disclosed methods and/or block diagrams described or claimed herein may also be stored on a computer-readable storage device.

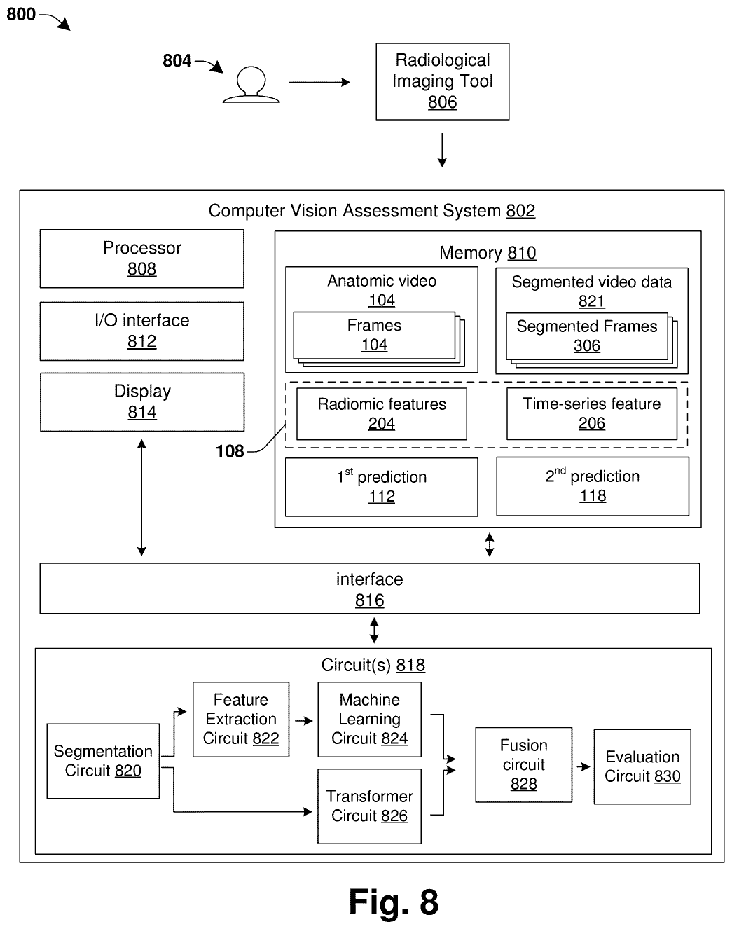

FIG. 8 illustrates a block diagram of some embodiments of a medical analysis system 800 comprising a computer vision assessment system configured to combine spatiotemporal hand-crafted features and a deep learning model to generate a medical prediction.

The medical analysis system 800 comprises a computer vision assessment system 802 configured to generate a medical prediction from a radiological video having a plurality of frames. In some embodiments, the computer vision assessment system 802 may be coupled to a radiological imaging tool 806 configured to act upon a patient 804. In some embodiment, the patient 804 may be a heart disease patient (e.g., a patient having or suspected of having heart disease). In some embodiments, the patient may be at increased risk of a major adverse cardiac event (MACE) such as a myocardial infraction, stroke, heart failure, revascularization, death, and/or the like. In some embodiments, the patient may also have chronic kidney disease (CKD). In some embodiments, the radiological imaging tool 806 may comprise and/or be an ultra-sound machine configured to perform an echocardiography. In other embodiments, the radiological imaging tool 806 may comprise and/or be an x-ray machine, a magnetic resonance imaging (MRI) machine, a positron emission tomography (PET) machine, a computed tomography (CT) machine, and/or the like.

The computer vision assessment system 802 comprises a processor 808 and a memory 810. The processor 808 can, in various embodiments, comprise circuitry such as, but not limited to, one or more single-core or multi-core processors. The processor 808 can include any combination of general-purpose processors and dedicated processors (e.g., graphics processors, application processors, etc.). The processor(s) 808 can be coupled with and/or can comprise memory (e.g., memory 810) or storage and can be configured to execute instructions stored in the memory 810 or storage to enable various apparatus, applications, or operating systems to perform operations and/or methods discussed herein.

Memory 810 can be configured to store data corresponding to an anatomic video 104 comprising a plurality of frames 105. The anatomic video 104 may correspond to the patient 804. Each of the plurality of frames 105 can have a plurality of pixels, each pixel having an associated intensity. In some embodiments, memory 810 can store video data from multiple anatomic videos as one or more training set(s) for training one or more machine learning models and/or one or more test sets for validating the one or more machine learning models.

The computer vision assessment system 802 also comprises an input/output (I/O) interface 812 (e.g., associated with one or more I/O devices), a display 814, a set of circuits 818, and an interface 816 that connects the processor 808, the memory 810, the I/O interface 812, and the set of circuits 818. The I/O interface 812 can be configured to transfer data between the memory 810, the processor 808, the circuits 818, and external devices, for example, the radiological imaging tool 806.

The set of circuits 818 can comprise a segmentation circuit 820, a feature extraction circuit 822, a machine learning circuit 824, a transformer circuit 826, a fusion circuit 828, and an evaluation circuit 830. In some embodiments, the set of circuits 818 may comprise computer code (e.g., one or more machine learning models) run on a processor. In other embodiments, the set of circuits 818 may comprise hardware components. The segmentation circuit 820 is configured to access the plurality of frames 105 and to generate segmented video data 821 comprising a plurality of segmented frames 306 that respectively identify a region of interest (ROI) (e.g., a left ventricle wall) within the plurality of frames 105. In some embodiments, the segmentation circuit 820 is configured to generate mask (e.g., a binary mask) stored in the memory 810. In some embodiments, the plurality of segmented frames 306 may be saved in the memory 810 as the plurality of frames 105.

In various embodiments, the feature extraction circuit 822 is configured to extract a plurality of hand-crafted features 108 from the plurality of frames 105. In some embodiment, the plurality of hand-crafted features 108 may comprise radiomic features 204 including texture features, morphological features, and/or the like. The feature extraction circuit 822 may be further configured to generate a time-series feature 206 using the plurality of radiomic features 204. In some embodiments, the plurality of hand-crafted features 108 may be stored in the memory 810.

In various embodiments, the machine learning circuit 824 may be configured to generate a first prediction 112 from the time-series feature 206. In some embodiments, the first prediction 112 may comprise a first numeric risk score.

The transformer circuit 826 is configured to operate upon the plurality of segmented frames 306 to generate a second prediction 118. In some embodiments, the second prediction 118 may comprise a second numeric risk score.

A fusion circuit 828 is configured to combine the first prediction 112 and the second prediction 118 to generate a biomarker. The evaluation circuit 830 is configured to utilize the biomarker to generate a medical prediction. In some embodiments, the medical prediction may relate to a MACE.

Therefore, the present disclosure relates to a computer vision assessment system that is configured to perform a spatiotemporal analysis of an anatomic video to generate a biomarker that can be used to generate a medical prediction.

In some embodiments, the present disclosure relates to a method that includes operating a first spatiotemporal model upon a plurality of frames of an anatomic video of a patient to determine a first prediction, the first spatiotemporal model being configured to determine the first prediction using a plurality of hand-crafted features extracted from the plurality of frames; operating a second spatiotemporal model, having one or more deep learning models, upon the plurality of frames of the anatomic video to determine a second prediction; and generating a medical prediction based upon a combination of the first prediction and the second prediction.

In other embodiments, the present disclosure relates to a non-transitory computer-readable medium storing computer-executable instructions that, when executed, cause a processor to perform operations, including accessing an echocardiography video of a heart of a patient having chronic kidney disease (CKD), the echocardiography video having a plurality of two-dimensional (2D) frames that are masked to identify a left ventricle wall (LVW) of the heart over a heartbeat cycle of the patient; operating upon the echocardiography video with a first spatiotemporal model that is configured to determine a first prediction from a first plurality of hand-crafted features extracted from the plurality of 2D frames; operating upon the echocardiography video with a second spatiotemporal model that is configured to determine a second prediction using deep learning, the second spatiotemporal model including a transformer model; combining the first prediction and the second prediction to generate a biomarker; and utilizing the biomarker to generate a medical prediction.

In yet other embodiments, the present disclosure relates to an apparatus including electronic memory configured to store an imaging data set including an anatomic video from a patient, the anatomic video having a plurality of frames; a first spatiotemporal model configured to operate on the anatomic video to extract a plurality of radiomic features and a time-series feature from the plurality of frames and to determine a first prediction from the time-series feature; a second spatiotemporal model configured to operate on the anatomic video to determine a second prediction using a series of transformer encoders; and an evaluation tool configured to generate a medical prediction using both the first prediction and the second prediction.

Embodiments discussed herein relate to training and/or employing machine learning models (e.g., unsupervised (e.g., clustering) or supervised (e.g., classifiers, etc.) models) to determine a medical prediction based on a combination of radiomic features and deep learning, based at least in part on features of medical imaging scans (e.g., MRI, CT, etc.) that are not perceivable by the human eye, and involve computation that cannot be practically performed in the human mind. As one example, machine learning classifiers and/or deep learning models as described herein cannot be implemented in the human mind or with pencil and paper. Embodiments thus perform actions, steps, processes, or other actions that are not practically performed in the human mind, at least because they require a processor or circuitry to access digitized images stored in a computer memory and to extract or compute features that are based on the digitized images and not on properties of tissue or the images that are perceivable by the human eye. Embodiments described herein can use a combined order of specific rules, elements, operations, or components that render information into a specific format that can then be used and applied to create desired results more accurately, more consistently, and with greater reliability than existing approaches, thereby producing the technical effect of improving the performance of the machine, computer, or system with which embodiments are implemented.

Examples herein can include subject matter such as an apparatus, including a digital whole slide scanner, a CT system, an MRI system, a personalized medicine system, a CADx system, a processor, a system, circuitry, a method, means for performing acts, steps, or blocks of the method, at least one machine-readable medium including executable instructions that, when performed by a machine (e.g., a processor with memory, an application-specific integrated circuit (ASIC), a field programmable gate array (FPGA), or the like) cause the machine to perform acts of the method or of an apparatus or system, according to embodiments and examples described.

References to “one embodiment”, “an embodiment”, “one example”, and “an example” indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

“Computer-readable storage device”, as used herein, refers to a device that stores instructions or data. “Computer-readable storage device” does not refer to propagated signals. A computer-readable storage device may take forms, including, but not limited to, non-volatile media, and volatile media. Non-volatile media may include, for example, optical disks, magnetic disks, tapes, and other media. Volatile media may include, for example, semiconductor memories, dynamic memory, and other media. Common forms of a computer-readable storage device may include, but are not limited to, a floppy disk, a flexible disk, a hard disk, a magnetic tape, other magnetic medium, an application specific integrated circuit (ASIC), a compact disk (CD), other optical medium, a random access memory (RAM), a read only memory (ROM), a memory chip or card, a memory stick, and other media from which a computer, a processor or other electronic device can read.

“Circuit”, as used herein, includes but is not limited to hardware, firmware, software in execution on a machine, or combinations of each to perform a function(s) or an action(s), or to cause a function or action from another logic, method, or system. A circuit may include a software controlled microprocessor, a discrete logic (e.g., ASIC), an analog circuit, a digital circuit, a programmed logic device, a memory device containing instructions, and other physical devices. A circuit may include one or more gates, combinations of gates, or other circuit components. Where multiple logical circuits are described, it may be possible to incorporate the multiple logical circuits into one physical circuit. Similarly, where a single logical circuit is described, it may be possible to distribute that single logical circuit between multiple physical circuits.

To the extent that the term “includes” or “including” is employed in the detailed description or the claims, it is intended to be inclusive in a manner similar to the term “comprising” as that term is interpreted when employed as a transitional word in a claim.

Throughout this specification and the claims that follow, unless the context requires otherwise, the words ‘comprise’ and ‘include’ and variations such as ‘comprising’ and ‘including’ will be understood to be terms of inclusion and not exclusion. For example, when such terms are used to refer to a stated integer or group of integers, such terms do not imply the exclusion of any other integer or group of integers.

To the extent that the term “or” is employed in the detailed description or claims (e.g., A or B) it is intended to mean “A or B or both”. When the applicants intend to indicate “only A or B but not both” then the term “only A or B but not both” will be employed. Thus, use of the term “or” herein is the inclusive, and not the exclusive use. See, Bryan A. Garner, A Dictionary of Modern Legal Usage 624 (2d. Ed. 1995).

While example systems, methods, and other embodiments have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and other embodiments described herein. Therefore, the invention is not limited to the specific details, the representative apparatus, and illustrative examples shown and described. Thus, this application is intended to embrace alterations, modifications, and variations that fall within the scope of the appended claims.

What is claimed is:

1. A method, comprising:
operating a first spatiotemporal model upon a plurality of frames of an anatomic video of a patient to determine a first prediction, wherein the first spatiotemporal model is configured to determine the first prediction using a plurality of hand-crafted features extracted from the plurality of frames;
operating a second spatiotemporal model, comprising one or more deep learning models, upon the plurality of frames of the anatomic video to determine a second prediction; and
generating a medical prediction based upon a combination of the first prediction and the second prediction.

2. The method of claim 1, wherein the plurality of frames comprise a plurality of segmented frames respectively masked to identify a left ventricle wall of a heart of the patient.

3. The method of claim 2, wherein the plurality of hand-crafted features are associated with spatiotemporal changes in the left ventricle wall of the heart.

4. The method of claim 1, wherein the plurality of frames span a time period that is equal to a heartbeat cycle of the patient.

5. The method of claim 1, further comprising:
adding or removing one or more frames from the anatomic video prior to operating upon the plurality of frames with the first spatiotemporal model or the second spatiotemporal model.

6. The method of claim 1, further comprising:
extracting a plurality of radiomic features for each of the plurality of frames of the anatomic video;
generating a time-series feature from the plurality of radiomic features; and
operating upon the time-series feature with a machine learning model to determine the first prediction.

7. The method of claim 6, wherein the plurality of radiomic features include one or more of a shape feature, a texture feature, and a first order statistic.

8. The method of claim 1, wherein the second spatiotemporal model comprises a first transformer encoder and a second transformer encoder arranged in series.

9. The method of claim 8,
wherein the first transformer encoder is configured to determine a spatial relationship between tokens from a same frame of the plurality of frames; and
wherein the second transformer encoder is configured to determine one or more temporal relationships between tokens from different ones of the plurality of frames.

10. The method of claim 1, further comprising:
generating a linear combination of the first prediction and the second prediction to determine the medical prediction.

11. A non-transitory computer-readable medium storing computer-executable instructions that, when executed, cause a processor to perform operations, comprising:
accessing an echocardiography video of a heart of a patient having chronic kidney disease (CKD), wherein the echocardiography video has a plurality of two-dimensional (2D) frames that are masked to identify a left ventricle wall (LVW) of the heart over a heartbeat cycle of the patient;
operating upon the echocardiography video with a first spatiotemporal model that is configured to determine a first prediction from a first plurality of hand-crafted features extracted from the plurality of 2D frames;
operating upon the echocardiography video with a second spatiotemporal model that is configured to determine a second prediction using deep learning, wherein the second spatiotemporal model comprises a transformer model;
combining the first prediction and the second prediction to generate a biomarker; and
utilizing the biomarker to generate a medical prediction.

12. The non-transitory computer-readable medium of claim 11, wherein the operations further comprise:
receiving the echocardiography video, wherein the echocardiography video has a first number of frames; and
adjusting a number of frames within the echocardiography video.

13. The non-transitory computer-readable medium of claim 11, wherein the operations further comprise:
extracting a plurality of radiomic features from respective ones of the plurality of 2D frames;
generating a time-series feature from the plurality of radiomic features extracted from respective ones of the plurality of 2D frames; and
determining the first prediction from the time-series feature.

14. The non-transitory computer-readable medium of claim 13, wherein the plurality of radiomic features include one or more of shape features, texture features, and first order statistics.

15. The non-transitory computer-readable medium of claim 11, wherein the second spatiotemporal model comprises:
a first transformer encoder; and
a second transformer encoder arranged downstream of the first transformer encoder.

16. An apparatus, comprising:
electronic memory configured to store an imaging data set comprising an anatomic video from a patient, wherein the anatomic video has a plurality of frames;
a first spatiotemporal model configured to operate on the anatomic video to extract a plurality of radiomic features and a time-series feature from the plurality of frames and to determine a first prediction from the time-series feature;
a second spatiotemporal model configured to operate on the anatomic video to determine a second prediction using a series of transformer encoders; and
an evaluation tool configured to generate a medical prediction using both the first prediction and the second prediction.

17. The apparatus of claim 16, wherein the time-series feature is determined from radiomic features extracted from multiple ones of the plurality of frames.

18. The apparatus of claim 16,
wherein the series of transformer encoders comprise a first transformer encoder configured to determine a spatial relationship between tokens from a same frame of the plurality of frames; and
wherein the series of transformer encoders comprise a second transformer encoder configured to determine one or more temporal relationships between tokens from different ones of the plurality of frames.

19. The apparatus of claim 16, wherein the patient has chronic kidney disease and the anatomic video is an echocardiography video of a heart of the patient.

20. The apparatus of claim 16, wherein the plurality of frames span a heartbeat cycle of the patient.
